(19) United States
(12) Patent Application Publication
(10) Pub. No.: US 2026/0250234 A1
(43) Pub. Date: Aug. 27, 2026

(54) PROPYLENE GLYCOL MONOMETHYL ETHER ACETATE PRODUCT

(71) Applicant: KH Neochem Co., Ltd., Chuo-ku, Tokyo (JP)

(72) Inventor: Kouji KURAMOTO, Yokkaichi-shi, Mie (JP)

(73) Assignee: KH Neochem Co., Ltd., Chuo-ku, Tokyo (JP)

(21) Appl. No.: 19/160,241

(22) PCT Filed: Dec. 7, 2023
(86) PCT No.: PCT/JP2023/043844

§ 371 Date: Aug. 27, 2025

(30) Foreign Application Priority Data
Mar. 3, 2023 (JP) 2023-033122

(51) Int. Cl. C07C 69/16

(52) U.S. Cl. CPC C07C 69/16

(57) ABSTRACT

A propylene glycol monomethyl ether acetate product containing propylene glycol 1-monomethyl ether 2-acetate, acetic acid, and water, wherein a content of the acetic acid is 5 ppm or more and 50 ppm or less based on 100% by mass of the propylene glycol monomethyl ether acetate product, and a content of the water is 20 ppm or more and 250 ppm or less based on 100% by mass of the propylene glycol monomethyl ether acetate product.

### TECHNICAL FIELD

The present invention relates to a propylene glycol monomethyl ether acetate product.

### BACKGROUND ART

Propylene glycol monomethyl ether acetate (also referred to as “PMA” hereinafter) is known as an organic solvent that is used for various purposes. PMA is manufactured by, for example, carrying out a direct esterification reaction of propylene glycol monomethyl ether (also referred to as “PM” hereinafter) with acetic acid under certain conditions (see, for example, Patent Literature 1).

### CITATION LIST

### Patent Literature

PTL 1: Chinese Unexamined Patent Application Publication No. 1515537

### SUMMARY OF INVENTION

### Technical Problem

It has been proved that when PMA obtained by the method described in Patent Literature 1 is stored for a long period of time and thereafter its composition is confirmed, purity of the PMA tends to lower.

The present invention has been made in view of the above point, and it is an object of the present invention to provide a propylene glycol monomethyl ether acetate product having excellent storage stability.

### Solution to Problem

The present inventor has found that a propylene glycol monomethyl ether acetate product containing specific components can solve the above problem, and thus completed the present invention.

That is to say, the present invention includes the following embodiments.

(1)

A propylene glycol monomethyl ether acetate product comprising:
propylene glycol 1-monomethyl ether 2-acetate,
acetic acid, and
water, wherein
a content of the acetic acid is 5 ppm or more and 50 ppm or less based on 100% by mass of the propylene glycol monomethyl ether acetate product, and
   a content of the water is 20 ppm or more and 250 ppm or less based on 100% by mass of the propylene glycol monomethyl ether acetate product.

(2)

The propylene glycol monomethyl ether acetate product according to (1), wherein a ratio of the content of the acetic acid to the content of the water, in terms of content of acetic acid (ppm)/content of water (ppm), is 0.01 or more and 1.50 or less.

(3)

The propylene glycol monomethyl ether acetate product according to (1) or (2), wherein when the propylene glycol monomethyl ether acetate product is subjected to the following test and thereafter subjected to gas chromatography analysis under the following conditions, and when a relative retention time of a peak of the propylene glycol 1-monomethyl ether 2-acetate is assumed to be 1.00, an area ratio of a peak appearing in the relative retention time range of 0.65 or more and 0.70 or less is 260 ppm or less:

### (Test)

In a borosilicate glass container, the propylene glycol monomethyl ether acetate product is heated to 80° C. and kept for 5 days in a nitrogen atmosphere,

### (Conditions of Gas Chromatography Analysis)

Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

(4)

The propylene glycol monomethyl ether acetate product according to any one of (1) to (3), wherein when the propylene glycol monomethyl ether acetate product is subjected to the following test and thereafter subjected to gas chromatography analysis under the following conditions, an area ratio of a peak of ethyl acetate is 12 ppm or less:

### (Test)

In a borosilicate glass container, the propylene glycol monomethyl ether acetate product is heated to 80° C. and kept for 5 days in a nitrogen atmosphere,

### (Conditions of Gas Chromatography Analysis)

Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

(5)

The propylene glycol monomethyl ether acetate product according to any one of (1) to (4), wherein when the propylene glycol monomethyl ether acetate product is subjected to the following test and thereafter subjected to gas chromatography analysis under the following conditions, and when a relative retention time of a peak of the propylene glycol 1-monomethyl ether 2-acetate is assumed to be 1.00, an area ratio of a peak appearing in the relative retention time range of 0.37 or more and 0.44 or less is 12 ppm or less:

### (Test)

In a borosilicate glass container, the propylene glycol monomethyl ether acetate product is heated to 80° C. and kept for 5 days in a nitrogen atmosphere,

### (Conditions of Gas Chromatography Analysis)

Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

(6)

The propylene glycol monomethyl ether acetate product according to any one of (1) to (5), wherein when the propylene glycol monomethyl ether acetate product is subjected to the following test and thereafter subjected to gas chromatography analysis under the following conditions, an area ratio of a peak of 2-acetoxy-1-propanol is 20 ppm or less:

### (Test)

In a borosilicate glass container, the propylene glycol monomethyl ether acetate product is heated to 80° C. and kept for 5 days in a nitrogen atmosphere,

### (Conditions of Gas Chromatography Analysis)

Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

(7)

The propylene glycol monomethyl ether acetate product according to any one of (1) to (6), which is used for manufacturing a semiconductor.

### Advantageous Effects of Invention

According to the present invention, a propylene glycol monomethyl ether acetate product having excellent storage stability can be provided.

### DESCRIPTION OF EMBODIMENTS

Hereinafter, an embodiment for carrying out the present invention (referred to as “the present embodiment” hereinafter) will be described in detail. The present embodiment is an example for explaining the present invention, and does not intend to limit the present invention to the following contents. The present invention can be carried out by being appropriately modified within the scope of its gist.

### <Propylene Glycol Monomethyl Ether Acetate Product>

The propylene glycol monomethyl ether acetate product (also referred to as “PMA product” hereinafter) of the present embodiment is a propylene glycol monomethyl ether acetate product comprising propylene glycol 1-monomethyl ether 2-acetate (also referred to as “PGMEA” hereinafter), acetic acid, and water, wherein a content of the acetic acid is 5 ppm or more and 50 ppm or less based on 100% by mass of the propylene glycol monomethyl ether acetate product, and a content of the water is 20 ppm or more and 250 ppm or less based on 100% by mass of the propylene glycol monomethyl ether acetate product (that is, the PMA product can be reworded as “PMA composition”). The propylene glycol monomethyl ether acetate product of the present embodiment is excellent in storage stability.

The reason why the PMA product of the present embodiment is excellent in storage stability is not necessarily clear. The reason is presumed to be, but not intended to be limited to, the following.

In long-term storage of a propylene glycol monomethyl ether acetate product, PGMEA is decomposed to generate impurities, and this is considered to be one of factors having an influence on the storage stability. Examples of causes of decomposition of a carboxylic acid ester such as PGMEA include hydrolysis. In general, in the presence of an acid and water, a carboxylic acid ester is hydrolyzed and becomes carboxylic acid and an alcohol. It is considered that when an acid content is high, the carboxylic acid ester tends to be more hydrolyzed. However, it is considered that, by adjusting the contents of acetic acid and water in the PMA product of the present embodiment to the above-mentioned ranges, a transesterification between alcohols, that may be produced by hydrolysis of PGMEA, and acetyl groups of PGMEA is suppressed, and as a result, hydrolysis of PGMEA is suppressed, which is different from the aforesaid tendency.

However, the above point is one that can be considered as a factor contributing to excellent storage stability of the PMA product of the present embodiment, and the mechanism of action of the present embodiment is not limited thereto.

The use purposes of the PMA product of the present embodiment are not particularly limited, but for example, the PMA product can be used for industrial purposes, such as inks, thinners, medical and agricultural chemicals, plasticizers, surfactants, polymer materials, lubricating oils, adhesives, cleaners, electronic materials, and coating materials.

Examples of the electronic materials include, but not limited to, a liquid crystal display (LCD) and a semiconductor device. The PMA product of the present embodiment is preferably used for manufacture of a semiconductor device because it is excellent in storage stability.

Specific examples of use purposes relating to the manufacture of a semiconductor device include, but not limited to, a cleaning liquid (e.g., processing liquid for cleaning a substrate surface or the like after resist stripping), a prewet liquid (e.g., processing liquid used before resist coating in order to reduce consumption of a resist solvent), a resist solvent (e.g., processing liquid for dissolving a photosensitizer or a resin), a developing solution (e.g., processing liquid for removing a soluble resin after exposure of a negative resist), and a stripping solution (e.g., processing liquid used for removing a cured resist after etching). From the viewpoint of prevention of product defects in the manufacture of a semiconductor device, the PMA product of the present embodiment is preferably used for a resist solvent, a prewet liquid, a solvent for an edge rinse used in negative resist coating (rinse for removing a resist in the outer periphery of a wafer) and a back rinse (rinse for removing a resist on a back surface of a wafer), a developing solution used in negative resist developing, a rinsing liquid used in rinsing after developing of a negative resist, a cleaning liquid or a rinsing liquid used for removing a resist after etching, etc.

(Propylene Glycol 1-monomethyl Ether 2-acetate)

The propylene glycol 1-monomethyl ether 2-acetate in the PMA product of the present embodiment can be identified and quantitatively determined by, for example, the following gas chromatography (also referred to as “GC” hereinafter) analysis.

### (Gas Chromatography Analysis)

Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL

The content of PGMEA in the PMA product of the present embodiment can be appropriately determined according to the use purpose of the PMA product of the present embodiment and is not particularly limited, but from the viewpoint that it is used for purposes requiring high purity, an area ratio of a peak based on the total peak area of a chart obtained from the results of the GC analysis is preferably 99.95% or more, more preferably 99.96% or more, and still more preferably 99.97% or more. In the present embodiment, “the total peak area” means a total of areas of all peaks appearing in a chart obtained from the results of the GC analysis. For example, when the relative retention time of a peak of PGMEA is assumed to be 1.00, the “all peaks” in the present embodiment can be specified as those that mean all of peaks appearing when the analysis is continued in a relative retention time of 0.14 to 2.95 and stopped.

### (Acetic Acid)

In the PMA product of the present embodiment, acetic acid is contained. From the viewpoint of storage stability, the content of acetic acid in the PMA product of the present embodiment is 5 ppm or more and 50 ppm or less, preferably 5 ppm or more and 45 ppm or less, and more preferably 5 ppm or more and 40 ppm or less, based on 100% by mass of the PMA product of the present embodiment.

The content can be measured based on the method described in Examples described later.

The content can be adjusted to the aforesaid range by, for example, appropriately adding acetic acid after base treatment described later is carried out. The content can also be adjusted to the aforesaid range by, for example, appropriately changing the conditions (e.g., treating time) of the base treatment and dehydration treatment.

### (Water)

In the PMA product of the present embodiment, water is contained. From the viewpoint of storage stability, the content of water in the PMA product of the present embodiment is 20 ppm or more and 250 ppm or less, preferably 23 ppm or more and 230 ppm of less, and more preferably 25 ppm or more and 230 ppm or less, based on 100% by mass of the PMA product of the present embodiment.

The content can be measured based on the method described in Examples described later.

The content can be adjusted to the aforesaid range by, for example, appropriately adding water after dehydration treatment described later is carried out. The content can also be adjusted to the aforesaid range by, for example, appropriately changing the conditions (e.g., treating time) of the dehydration treatment.

### (Relationship Between Content of Acetic Acid and Content of Water)

From the viewpoint of storage stability in the present embodiment, a ratio of a content of acetic acid to a content of water in the PMA product of the present embodiment is preferably 0.01 or more and 1.50 or less, more preferably 0.02 or more and 1.25 or less, and still more preferably 0.05 or more and 1.10 or less, in terms of content (ppm) of acetic acid based on 100% by mass of the PMA product of the present embodiment/content (ppm) of water based on 100% by mass of the PMA product of the present embodiment.

### (Component A)

When the PMA product of the present embodiment is subjected to the following test and thereafter subjected to the aforesaid GC analysis, and when a relative retention time of a peak of propylene glycol 1-monomethyl ether 2-acetate is assumed to be 1.00, an area ratio of a peak (substance corresponding to this peak is also referred to as “component A”) appearing in the relative retention time range of 0.65 or more and 0.70 or less is preferably 260 ppm or less, more preferably 220 ppm or less, and sill more preferably 200 ppm or less, from the viewpoint of storage stability.

### (Test)

In a borosilicate glass container, the propylene glycol monomethyl ether acetate product is heated to 80° C. and kept for 5 days in a nitrogen atmosphere.

The area ratio can be measured based on the method described in Examples described later.

The area ratio can be adjusted to the aforesaid range by, for example, appropriately adding acetic acid and/or water after base treatment and/or dehydration treatment described later is carried out. The area ratio can also be adjusted to the aforesaid range by, for example, appropriately changing the conditions (e.g., treating time) of the base treatment and/or the dehydration treatment.

### (Ethyl Acetate)

When the PMA product of the present embodiment is subjected to the aforesaid test and thereafter subjected to the aforesaid GC analysis, an area ratio of a peak of ethyl acetate is preferably 12 ppm or less, more preferably 10 ppm or less, and still more preferably 9 ppm or less, from the viewpoint of storage stability.

The area ratio can be measured based on the method described in Examples described later.

The area ratio can be adjusted to the aforesaid range by, for example, appropriately adding acetic acid and/or water after base treatment and/or dehydration treatment described later is carried out. The area ratio can also be adjusted to the aforesaid range by, for example, appropriately changing the conditions (e.g., treating time) of the base treatment and/or the dehydration treatment.

### (Component B)

When the PMA product of the present embodiment is subjected to the aforesaid test and thereafter subjected to the aforesaid GC analysis, and when a relative retention time of a peak of propylene glycol 1-monomethyl ether 2-acetate is assumed to be 1.00, an area ratio of a peak (substance corresponding to this peak is also referred to as “component B”) appearing in the relative retention time range of 0.37 or more and 0.44 or less is preferably 12 ppm or less, more preferably 10 ppm or less, and still more preferably 8 ppm or less, from the viewpoint of storage stability.

The area ratio can be measured based on the method described in Examples described later.

The area ratio can be adjusted to the aforesaid range by, for example, appropriately adding acetic acid and/or water after base treatment and/or dehydration treatment described later is carried out. The area ratio can also be adjusted to the aforesaid range by, for example, appropriately changing the conditions (e.g., treating time) of the base treatment and/or the dehydration treatment.

### 2-Acetoxy-1-propanol

When the PMA product of the present embodiment is subjected to the aforesaid test and thereafter subjected to the aforesaid GC analysis, an area ratio of a peak of 2-acetoxy-1-propanol is preferably 20 ppm or less, more preferably 18 ppm or less, and still more preferably 16 ppm or less, from the viewpoint of storage stability.

The area ratio can be measured based on the method described in Examples described later.

The area ratio can be adjusted to the aforesaid range by, for example, appropriately adding acetic acid and/or water after base treatment and/or dehydration treatment described later is carried out. The area ratio can also be adjusted to the aforesaid range by, for example, appropriately changing the conditions (e.g., treating time) of the base treatment and/or the dehydration treatment.

When the PMA product of the present embodiment is subjected to the aforesaid test and thereafter subjected to the aforesaid GC analysis, an area ratio of a peak of PGMEA is preferably 99.93% or more, more preferably 99.94% or more, and still more preferably 99.95% or more, of the total peak area, from the viewpoint of storage stability.

The area ratio can be measured based on the method described in Examples described later.

The area ratio can be adjusted to the aforesaid range by, for example, appropriately adding acetic acid and/or water after base treatment and/or dehydration treatment described later is carried out. The area ratio can also be adjusted to the aforesaid range by, for example, appropriately changing the conditions (e.g., treating time) of the base treatment and/or the dehydration treatment.

### <Method for Manufacturing Propylene Glycol Monomethyl Ether Acetate Product>

The method for manufacturing the PMA product of the present embodiment is not particularly limited, but the following method (also referred to as “manufacturing method A” hereinafter) is preferable. The manufacturing method A preferably includes a step (a) of obtaining a first product containing PGMEA, a step (b) of distilling the first product to obtain a second product, a step (c) of subjecting the second product to base treatment to obtain a third product, a step (d) of distilling the third product to obtain a fourth product, and a step (e) of subjecting the fourth product to dehydration treatment to obtain a PMA product. The amounts of acetic acid and water in the PMA product can be adjusted by the step (c) and the step (e), and this method may further include a step (f) of adding acetic acid and/or water to a product obtained through the step (e) to obtain a PMA product.

### (Step (a))

In the step (a), a first product containing PGMEA is obtained. The step (a) can include operations for manufacturing PGMEA based on a conventionally known method. Examples of the method for manufacturing PGMEA include, but not limited to, a method described in Chinese Unexamined Patent Application Publication No. 1515537. Specifically, PGMEA can be manufactured by carrying out a direct esterification reaction of PM with acetic acid. In the first product, raw materials that can be used in the reaction for manufacturing PGMEA, a catalyst, a by-product, etc. may be contained.

### (Step (b))

In the step (b), the first product is distilled to obtain a second product. Examples of specific operations for distillation include, but not limited to, atmospheric distillation and vacuum distillation, and such distillation may be repeatedly carried out. In this step, raw materials that can be used in the reaction for manufacturing PGMEA, a catalyst, a by-product, etc., which can be contained in the first product, can be removed. The conditions of distillation are not particularly limited, but for example, distillation can be carried out by reference to the conditions, etc. described in Chinese Unexamined Patent Application Publication No. 1515537. The amounts of acetic acid and water contained in the second product may be more than 50 ppm and more than 250 ppm, respectively, each being based on 100% by mass of the second product.

### (Step (c))

In the step (c), the second product is subjected to base treatment to obtain a third product. The conditions of the base treatment are preferably conditions under which the amount of acetic acid contained in the second product can be reduced, but they are not particularly limited, and for example, the second product is subjected to base treatment with KYOWAAD™ 500 manufactured by Kyowa Chemical Industry Co., Ltd. At this time, the amount of acetic acid contained in the third product can be adjusted by, for example, controlling the treating time.

### (Step (d))

In the step (d), the third product is distilled to obtain a fourth product. Examples of specific operations for distillation include, but not limited to, vacuum distillation, and such distillation may be repeatedly carried out. In this step, inorganic matters that can be contained in the third product can be removed. The conditions of distillation are not particularly limited, but for example, distillation can be carried out by reference to the conditions, etc. described in Chinese Unexamined Patent Application Publication No. 1515537.

### (Step (e))

In the step (e), the fourth product is subjected to dehydration treatment to obtain a PMA product. The conditions of the dehydration treatment are preferably conditions under which the amount of water contained in the fourth product can be reduced, but they are not particularly limited, and for example, the fourth product is subjected to dehydration treatment by nitrogen bubbling. At this time, the amount of water contained in the PMA product can be adjusted by, for example, controlling the treating time.

### (Step (f))

In the step (f), the amounts of acetic acid and/or water in the PMA product can be adjusted by adding acetic acid and/or water to a product obtained through the step (e). The amounts of acetic acid and/or water added are not particularly limited, but for example, they can be determined based on differences between a content C1 of acetic acid and a content C2 of water in the desired PMA product and a content C1′ of acetic acid and a content C2′ of water in the product obtained through the step (e). The contents can be each measured based on the method described in Examples described later.

### EXAMPLES

Hereinafter, the present embodiment will be described in more detail based on Examples. The present embodiment is not limited to the Examples.

### Example 1

### (Step (a))

A first product containing PGMEA was synthesized by reference to the method described in Chinese Unexamined Patent Application Publication No. 1515537. That is to say, a first product containing PGMEA was obtained by a direct esterification reaction of PM with acetic acid.

### (Step (b))

Subsequently, the first product containing PGMEA was led to a distillation column, and atmospheric distillation was carried out. That is to say, the reflux ratio was set to 1 to 9, and unreacted raw materials, etc. were distilled away first, followed by recovering a fraction containing PGMEA.

Subsequently, the fraction containing PGMEA obtained by atmospheric distillation was led to a distillation column, and vacuum distillation was carried out. That is to say, the degree of vacuum in the distillation column was set to −0.08 MPa (gauze pressure), the temperature was controlled such that the temperature in the distillation column was 110° C. or lower, the reflux ratio was set to 1 to 6, and a fraction of a column top (temperature: 98 to 100° C.) was recovered as a second product. The content of propylene glycol 1-monomethyl ether 2-acetate in the second product obtained was 99.97% in terms of an area ratio of a peak based on the total peak area of a chart obtained from the results of GC analysis. The amounts of acetic acid and water contained in the second product were 90 ppm and 300 ppm, respectively, each being based on 100% by mass of the second product.

The contents of PGMEA, acetic acid and water contained in the second product were confirmed by the following methods. That is to say, the contents of PGMEA and acetic acid were measured by GC analysis under the following conditions. The content of water was measured with a Karl Fischer moisture meter (product name “AQ-2200A”, manufactured by HIRANUMA Co., Ltd., Karl Fischer coulometric titration method).

### (Gas Chromatography Analysis)

Analysis equipment: manufactured by Shimadzu Corporation, Nexis GC-2030
Analytical column: manufactured by Agilent Technologies, Inc., DB-WAX (column with polyethylene glycol as a stationary phase and with length 30 m×inner diameter 0.25 mm×film thickness 0.25 μm)
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL

In the above GC analysis, the content of PGMEA was calculated as an area percentage of a peak of PGMEA based on the total peak area of a chart obtained from the results of GC analysis. The content of acetic acid was calculated by an internal standard method (internal standard substance: biphenyl) through GC analysis.

Also in the following examples and comparative examples, the contents of acetic acid and water were confirmed in the same manner as above.

### (Step (c))

To the second product (100% by mass) obtained above, 0.05% by mass of KYOWAAD™ 500 manufactured by Kyowa Chemical Industry Co., Ltd. was added, and they were stirred for 60 minutes to remove acetic acid by adsorption. This was filtered through a PTFE membrane filter (model: T020A047A, pore size: 0.20 μm) manufactured by the ADVANTEC Group to obtain a third product.

### (Step (d))

Subsequently, the third product was led to a distillation column, the degree of vacuum in the distillation column was set to −0.08 MPa (gauze pressure), the temperature in the distillation column was controlled to 110° C. or lower, the reflux ratio was set to 1 to 6, and a fraction of a column top (98 to 100° C.) was obtained as a fourth product.

### (Step (e))

Into the fourth product, nitrogen having been filtered through a Kinoshita type ball filter (model number: 501G-1, filter diameter: 10 mm, filter pore size: 100 to 120 μm) manufactured by Kinoshita Rika Kogyo Co., Ltd. was bubbled at 3 to 5 L/min for 90 minutes to carry out dehydration treatment, thereby obtaining a PMA product of Example 1. The amounts of acetic acid and water contained in the PMA product were 5 ppm and 55 ppm, respectively, each being based on 100% by mass of the PMA product.

Subsequently, the PMA product was subjected to the following test. That is to say, in a 110 mL borosilicate glass container, the PMA product was placed, and nitrogen was sealed in the container, thereafter the container was tightly sealed, and the PMA product was heated using a thermostat (product name “ST-110B1” manufactured by ESPEC CORP.) and kept at 80° C. for 5 days.

The PMA product after the test was subjected to gas chromatography analysis under the following conditions.

### (Conditions of Gas Chromatography Analysis)

Analysis equipment: Nexis GC-2030 (manufactured by Shimadzu Corporation)
Analytical column: DB-WAX (manufactured by Agilent Technologies, Inc., column with polyethylene glycol as a stationary phase and with length 30 m×inner diameter 0.25 mm×film thickness 0.25 μm)
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL

A GC chart B obtained by the GC analysis after the above test and a GC chart A obtained by the GC analysis before the test were compared, then a substance whose peak area ratio was less than 5 ppm in the chart A and whose peak area ratio was 5 ppm or more in the chart B was considered to be a substance having a significant influence on the storage stability of the PMA product, and the amounts of them were evaluated. That is to say, when the relative retention time of a peak of propylene glycol 1-monomethyl ether 2-acetate was assumed to be 1.00, an area ratio of a peak appearing when the relative retention time was 0.66 and an area ratio of a peak appearing when the relative retention time was 0.40 were 135 area ppm and 7 area ppm, respectively, in the GC chart B. In the GC chart B, an area ratio of a peak of ethyl acetate and an area ratio of a peak of 2-acetoxy 1-propanol were 8 area ppm and 15 area ppm, respectively. Details of the analytical results are shown in Table 1.

### Example 2

A PMA product of Example 2 was obtained in the same manner as in Example 1, except that the stirring time of the step (c) in Example 1 was changed to 45 minutes, and the bubbling time of the step (e) in Example 1 was changed to 100 minutes. The PMA product was subjected to the same test as in Example 1, and thereafter subjected to the same gas chromatography analysis as in Example 1. Details of the analytical results are shown in Table 1.

### Example 3

A PMA product of Example 3 was obtained in the same manner as in Example 1, except that the stirring time of the step (c) in Example 1 was changed to 40 minutes, and the bubbling time of the step (e) in Example 1 was changed to 30 minutes. The PMA product was subjected to the same test as in Example 1, and thereafter subjected to the same gas chromatography analysis as in Example 1. Details of the analytical results are shown in Table 1.

### Example 4

A PMA product of Example 4 was obtained in the same manner as in Example 1, except that the stirring time of the step (c) in Example 1 was changed to 25 minutes, and the bubbling time of the step (e) in Example 1 was changed to 80 minutes. The PMA product was subjected to the same test as in Example 1, and thereafter subjected to the same gas chromatography analysis as in Example 1. Details of the analytical results are shown in Table 1.

### Comparative Example 1

A PMA product of Comparative Example 1 was obtained in the same manner as in Example 1, except that the stirring time of the step (c) in Example 1 was changed to 60 minutes, and the bubbling time of the step (e) in Example 1 was changed to 120 minutes. The PMA product was subjected to the same test as in Example 1, and thereafter subjected to the same gas chromatography analysis as in Example 1. Details of the analytical results are shown in Table 1.

### Comparative Example 2

A PMA product of Comparative Example 2 was obtained in the same manner as in Example 1, except that the stirring time of the step (c) in Example 1 was changed to 45 minutes, and the bubbling time of the step (e) in Example 1 was changed to 120 minutes. The PMA product was subjected to the same test as in Example 1, and thereafter subjected to the same gas chromatography analysis as in Example 1. Details of the analytical results are shown in Table 1.

### Comparative Example 3

A PMA product of Comparative Example 3 was obtained in the same manner as in Example 1, except that the stirring time of the step (c) in Example 1 was changed to 5 minutes, and the bubbling time of the step (e) in Example 1 was changed to 110 minutes. The PMA product was subjected to the same test as in Example 1, and thereafter subjected to the same gas chromatography analysis as in Example 1. Details of the analytical results are shown in Table 1.

TABLE 1

| | | Content based on | | | | | | | |
|---|---|---|---|---|---|---|---|---|---|
| | Peak area ratio | 100% by mass of | | | Area ratio of peak of each component after test | | | | |
| | before test | PMA product | | Acetic acid | | Component | Ethyl | Component | 2-Acetoxy- |
| | PGMEA | Acetic acid | Water | content/water | PGMEA | A | acetate | B | 1-propanol |
| | (area %) | (ppm) | (ppm) | content | (area %) | (area ppm) | (area ppm) | (area ppm) | (area ppm) |
| Ex. 1 | 99.970 | 5 | 55 | 0.09 | 99.952 | 135 | 8 | 7 | 15 |
| Ex. 2 | 99.970 | 25 | 40 | 0.63 | 99.938 | 203 | 9 | 8 | 16 |
| Ex. 3 | 99.969 | 30 | 230 | 0.13 | 99.950 | 181 | 6 | 7 | 13 |
| Ex. 4 | 99.970 | 50 | 70 | 0.71 | 99.939 | 189 | 9 | 8 | 15 |
| Comp. Ex. 1 | 99.970 | 5 | 15 | 0.33 | 99.926 | 277 | 13 | 14 | 22 |
| Comp. Ex. 2 | 99.970 | 25 | 15 | 1.67 | 99.915 | 309 | 27 | 25 | 45 |
| Comp. Ex. 3 | 99.971 | 75 | 25 | 3.00 | 99.896 | 463 | 28 | 28 | 47 |

(Note 1)
Component A is a substance corresponding to a peak appearing in the relative retention time range of 0.65 or more and 0.70 or less when the relative retention time of a peak of PGMEA is assumed to be 1.00.
(Note 2)
Component B is a substance corresponding to a peak appearing in the relative retention time range of 0.37 or more and 0.44 or less when the relative retention time of a peak of PGMEA is assumed to be 1.00.

1. A propylene glycol monomethyl ether acetate product comprising: propylene glycol 1-monomethyl ether 2-acetate, acetic acid, and water, wherein a content of the acetic acid is 5 ppm or more and 50 ppm or less based on 100% by mass of the propylene glycol monomethyl ether acetate product, and a content of the water is 20 ppm or more and 250 ppm or less based on 100% by mass of the propylene glycol monomethyl ether acetate product.

2. The propylene glycol monomethyl ether acetate product according to claim 1, wherein a ratio of the content of the acetic acid to the content of the water, in terms of content of acetic acid (ppm)/content of water (ppm), is 0.01 or more and 1.50 or less.

3. The propylene glycol monomethyl ether acetate product according to claim 1, wherein when the propylene glycol monomethyl ether acetate product is subjected to the following test and thereafter subjected to gas chromatography analysis under the following conditions, and when a relative retention time of a peak of the propylene glycol 1-monomethyl ether 2-acetate is assumed to be 1.00, an area ratio of a peak appearing in the relative retention time range of 0.65 or more and 0.70 or less is 260 ppm or less:
(Test)
In a borosilicate glass container, the propylene glycol monomethyl ether acetate product is heated to 80° C. and kept for 5 days in a nitrogen atmosphere,
(Conditions of gas chromatography analysis)
Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

4. The propylene glycol monomethyl ether acetate product according to claim 1, wherein when the propylene glycol monomethyl ether acetate product is subjected to the following test and thereafter subjected to gas chromatography analysis under the following conditions, an area ratio of a peak of ethyl acetate is 12 ppm or less:
(Test)
In a borosilicate glass container, the propylene glycol monomethyl ether acetate product is heated to 80° C. and kept for 5 days in a nitrogen atmosphere,
(Conditions of gas chromatography analysis)
Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

5. The propylene glycol monomethyl ether acetate product according to claim 1, wherein when the propylene glycol monomethyl ether acetate product is subjected to the following test and thereafter subjected to gas chromatography analysis under the following conditions, and when a relative retention time of a peak of the propylene glycol 1-monomethyl ether 2-acetate is assumed to be 1.00, an area ratio of a peak appearing in the relative retention time range of 0.37 or more and 0.44 or less is 12 ppm or less:
(Test)
In a borosilicate glass container, the propylene glycol monomethyl ether acetate product is heated to 80° C. and kept for 5 days in a nitrogen atmosphere,
(Conditions of gas chromatography analysis)
Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

6. The propylene glycol monomethyl ether acetate product according to claim 1, wherein when the propylene glycol monomethyl ether acetate product is subjected to the following test and thereafter subjected to gas chromatography analysis under the following conditions, an area ratio of a peak of 2-acetoxy-1-propanol is 20 ppm or less:
(Test)
In a borosilicate glass container, the propylene glycol monomethyl ether acetate product is heated to 80° C. and kept for 5 days in a nitrogen atmosphere,
(Conditions of gas chromatography analysis)
Analytical column: column with polyethylene glycol as a stationary phase and with length 30 m×inner diameter 0.25 mm×film thickness 0.25 μm
Heating conditions: maintained at 50° C. for 10 minutes and thereafter heated up to 250° C. at 5° C./min
Sample introduction temperature: 250° C.
Carrier gas: nitrogen
Gas flow rate of column: 1.0 mL/min
Detector and detection temperature: hydrogen flame ionization detector, 250° C.
Control mode: column flow rate
Split ratio: 50:1
Injection volume: 2.0 μL.

7. The propylene glycol monomethyl ether acetate product according to claim 1, which is used for manufacturing a semiconductor.
